(19) United States
(12) Patent Application Publication
(10) Pub. No.: US 2025/0032322 A1
(43) Pub. Date: Jan. 30, 2025

(54) METHOD AND APPARATUS FOR PROVIDING NEGATIVE PRESSURE TO A NEGATIVE PRESSURE WOUND THERAPY BANDAGE

(71) Applicant: Smith & Nephew, Inc., Memphis, TN (US)

(72) Inventors: John Buan, Maple Grove, MN (US); Richard Willems, Big Lake, MN (US)

(21) Appl. No.: 18/805,986

(22) Filed: Aug. 15, 2024

Related U.S. Application Data
(63) Continuation of application No. 17/555,195, filed on Dec. 17, 2021, now Pat. No. 12,097,095.
Application No. 17/555,195 is a continuation of application No. 16/234,241, filed on Dec. 27, 2018, now abandoned.
Application No. 16/234,241 is a continuation of application No. 14/737,387, filed on Jun. 11, 2015, now Pat. No. 10,300,178.
Application No. 14/737,387 is a continuation of application No. 13/299,783, filed on Nov. 18, 2011, now Pat. No. 9,067,003.
(60) Provisional application No. 61/490,118, filed on May 26, 2011.

(51) Int. Cl. A61F 13/05; A61F 13/00; A61M 1/00

(52) U.S. Cl. CPC A61F 13/05; A61M 1/74; A61M 1/75; A61M 1/96; A61F 2013/00174; A61F 2013/00536; A61M 2205/33; A61M 2210/04

(57) ABSTRACT

A method for providing a negative pressure wherein a pump is cycled on and off to achieve a target negative pressure, which is set slightly lower than the therapeutic negative pressure. A device determines the actual pressure by averaging samples which may occur at a different rate than the pump cycle.

### CROSS REFERENCE TO RELATED APPLICATIONS

This application is a continuation of U.S. patent application Ser. No. 17/555,195, filed Dec. 17, 2021, which is a continuation of U.S. patent application Ser. No. 16/234,241, filed Dec. 27, 2018, which is a continuation of U.S. patent application Ser. No. 14/737,387, filed Jun. 11, 2015 and issued as U.S. Pat. No. 10,300,178, which is a continuation of U.S. patent application Ser. No. 13/299,783, filed Nov. 18, 2011 and issued as U.S. Pat. No. 9,067,003, which claims priority to U.S. Provisional Application No. 61/490,118, filed May 26, 2011, titled “METHOD FOR PROVIDING NEGATIVE PRESSURE TO A NEGATIVE PRESSURE WOUND THERAPY BANDAGE.” The disclosure of each of these prior applications is hereby incorporated by reference in its entirety.

### FIELD OF THE INVENTION

The invention relates to a method for providing negative pressure to a negative pressure wound therapy bandage.

### BACKGROUND OF THE INVENTION

Negative pressure wound therapy is one method that is used to treat certain wounds or sores on people. In general the treatment includes, a bandage being placed over a wound site, and connected to a pumping device. The pumping device provides suction, creating a negative pressure under the bandage at the wound site. Exudates and other materials are removed from the wound site, allowing the wound to heal faster than under ambient pressure.

The pumping device includes, amongst other things, a pump. Typically, a user selects (or otherwise enters) an appropriate therapeutic pressure on the pumping device that correlates to the specific negative pressure treatment for that patient. It is important that the pump and pumping device achieve the therapeutic pressure that is to be used in association with the negative pressure wound therapy quickly and correctly.

The speed at which the pump can achieve the therapeutic pressure is important because the speed can detrimentally effect the treatment. Moreover, the ability for the pump to recognize that the therapeutic pressure has been achieved is important to avoid applying too little or too much negative pressure. Thus, for a pump device, the speed and the accuracy at which it obtains the therapeutic pressure is an important characteristic.

In order to accurately and quickly achieve the therapeutic pressure, some pumps are left on continuously. In some devices this can be problematic. For example, some pumps can quickly achieve the therapeutic pressure; however, the pump and system tends to overshoot the therapeutic pressure value. This requires additional valves and other components that allow the pumping device to release negative pressure until the appropriate negative pressure is obtained.

Moreover, leaving the pump on continuously can act as a power drain, and for pumping devices that operate on batteries or other portable power sources, this can negatively impact the life time of the power source.

The present invention is directed to resolving these and other matters.

### SUMMARY OF THE INVENTION

In one embodiment of the invention, the invention is directed towards a method for providing negative pressure to a negative pressure wound therapy bandage by providing a pumping device having a pump and setting a therapeutic negative pressure on the pumping device. The pumping device determines a target negative pressure associated with the therapeutic negative pressure and the target negative pressure is less than the therapeutic negative pressure (as discussed herein, “less than” means it is less negative, or a lower amount of a negative pressure, in other words, closer to zero). The pumping device is connected to a negative pressure wound therapy bandage. The target negative pressure is achieved by cycling the pump on and off to create a negative pressure in the negative pressure wound therapy bandage, obtaining a plurality of samples of the negative pressure in the negative pressure wound therapy bandage, each sample being obtained at a first set time interval, calculating an average of a predetermined number of consecutive samples, comparing the average to the target negative pressure, and, continuing to cycle the pump on and off until the average is greater than the target pressure.

The method may also include after cycling the pump on and off until the average of samples is greater than the target pressure after the complete off cycle, sampling the negative pressure in the negative pressure wound therapy bandage at a second set time interval greater than the first set time interval.

In another embodiment of the invention, the sampling of the negative pressure in the negative pressure wound therapy bandage at the second set time interval begins when the pump has been off for a predetermined amount of time.

The predetermined amount of time may be 300 milliseconds.

The second set time interval may be 1000 milliseconds and the first set time interval is 100 milliseconds.

In an embodiment, the cycling of the pump on and off to create negative pressure in the negative pressure wound therapy bandage on is performed by turning the pump on for 80 milliseconds and then turning the pump off for 80 milliseconds.

In some embodiments of the present invention, the therapeutic negative pressure is 125 mmHg and the target negative pressure is approximately 115 mmHg.

In some embodiments of the present invention, the therapeutic negative pressure is 90 mmHg and the target negative pressure is approximately 85 mmHg.

In some embodiments of the present invention, the therapeutic negative pressure is 60 mmHg and the target negative pressure is approximately 54 mmHg.

In some embodiments of the present invention, the therapeutic negative pressure is 40 mmHg and the target negative pressure is approximately 34 mmHg.

In another embodiment of the invention, the invention is for a method for providing negative pressure to a negative pressure wound therapy bandage by providing a pumping device having a pump and setting a therapeutic negative pressure on the pumping device. The pumping device determines a target negative pressure associated with the therapeutic negative pressure and the target negative pressure is less than the therapeutic negative pressure. The pumping device is connected to a negative pressure wound therapy bandage. The target negative pressure is achieved by cycling the pump on and off for a first predetermined amount of time to create a negative pressure in the negative pressure wound therapy bandage, obtaining a plurality of samples of the negative pressure in the negative pressure wound therapy bandage, each sample being obtained at a first set time interval, calculating an average of a predetermined number of consecutive samples, comparing the average to the target negative pressure, and, if the average is not greater than the target negative pressure, repeating the steps of cycling the pump on and off, obtaining a plurality of samples, calculating an average and comparing the average, until the average is greater than the target negative pressure.

In some embodiments the comparing of the average occurs at a rate faster than the rate of calculating the average.

In some embodiments, if the average is greater than the target negative pressure, the target negative pressure is maintained by obtaining a plurality of second samples of the negative pressure in the negative pressure wound therapy bandage at a second set time interval begin greater than the first time interval, and, comparing each second sample to the target negative pressure, and, repeating the steps of obtaining and comparing if a second sample is greater than the target negative pressure.

In some embodiments, if a second sample is not greater than the target negative pressure, the step of achieving the target negative pressure by: cycling the pump on and off; obtaining a plurality of samples; calculating an average; and, comparing the average until the average is greater than the target negative pressure, is repeated.

In the various embodiments of the present invention, the therapeutic negative pressure may be selected from the group consisting of: 40 mmHg; 60 mmHg; 90 mmHg; and, 125 mmHg.

An invention according to one or more of the disclosed embodiments allows the pump to achieve the therapeutic negative pressure/target negative pressure reasonably quickly and limit exceeding the therapeutic negative pressure/target negative pressure as much as practical.

Moreover, at least one of the embodiments also provides a reliable method to detect when the bandage pressure was being “topped-up” too frequently due to a leak (i.e., often returning to cycle the pump on and off to increase negative pressure). It is expected that the system will leak a small amount over time, for example, due to the imperfect plumbing connections in the system. A leak rate of one “top-up” per minute is normal. However, a leak rate four times that is cause for a minor leak to be declared. Such a leak rate is still within the bounds of the system to handle long term but the user should be informed so that it is corrected and system battery life is extended. It is also desirable to clear the fault as soon as it appears that the leaking has returned to normal levels.

It is to be understood that the aspects and objects of the present invention described above may be combinable and that other advantages and aspects of the present invention will become apparent upon reading the following description of the drawings and detailed description of the invention.

### BRIEF DESCRIPTION OF THE DRAWINGS

The present invention will become more fully apparent from the following description and appended claims, taken in conjunction with the accompanying drawings. Understanding that the accompanying drawings depict only typical embodiments, and are, therefore, not to be considered to be limiting of the scope of the present disclosure, the embodiments will be described and explained with specificity and detail in reference to the accompanying drawings as provided below.

FIG. 1 is a front perspective view of a device used in association with one or more embodiments of the present invention.

FIG. 2 is a front side cutaway view of a device provided according to one or more embodiments of the present invention.

FIG. 3 is a flow chart of a method according to one or more embodiments of the present invention.

### DETAILED DESCRIPTION OF THE DRAWINGS

While this invention is susceptible of embodiment in many different forms, there is shown in the drawings and will herein be described in detail one or more embodiments with the understanding that the present disclosure is to be considered as an exemplification of the principles of the invention and is not intended to limit the invention to the embodiments illustrated.

Reference throughout this description to features, advantages, objects or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

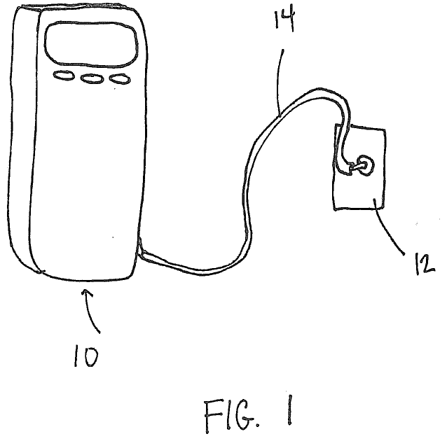

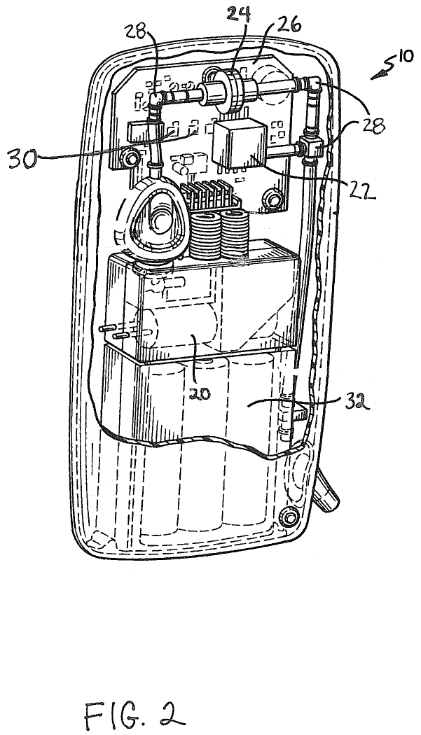

As shown in the attached FIGS. 1 and 2, the present invention is directed towards a method used with a pumping device 10 having a pump 20. The pumping device 10 generally includes a pressure sensor 22, a check valve 24, a control circuit 26 (with microcontroller), internal plumbing 28, LEDs 30 and a power source 32.

In order to provide negative pressure to a wound, the pumping device 10 is connected to a negative pressure wound therapy bandage 12, typically with tubing 14.

In order to achieve a target negative pressure the pumping device 10 turns the pump 20 on for short amounts of time when the pressure is below the target pressure.

The pump device 10 typically operates as follows: a microcontroller turns the pump 20 on via a digital output; the pump 20 starts producing a vacuum; the vacuum is converted to a voltage by a pressure sensor 22; the voltage is converted by an analog to digital converter (“the AID”) to a standard, recognized pressure engineering unit; and, the microcontroller compares the monitored pressure to the target pressure.

In order to achieve the target negative pressure in a short amount of time and avoid overshooting the target negative pressure, the pump 20 is cycled. The duty cycle, the percentage of pump on time relative to pump off time, after examining several pumps in action, can be constant for a variety of pumps 20. How much vacuum the pump 20 can draw in a short time period will influence the pump on time. Moreover, bandage 14 and plumbing 28 elasticity and how quickly the pressure sensor 22 and the AID can take a measurement will influence the pump off time.

In one embodiment, the pressure sensor 22 measurement time is 20 milliseconds; while the AID measurement time is 16 microseconds. Therefore, since a change in pressure must first be converted by the pressure sensor 22 and then the AID, the minimum measurement time is 20.016 milliseconds.

It is believed to be advantageous to average readings before they are used to mitigate occasional measurement system anomalies. The number of samples in the average, on the one hand, when it tends to be low, do some anomaly mitigation but also remain the most responsive to quick changes in the parameter they are measuring. The best anomaly mitigation appears to occur when the number of samples is high, but then the averaging is not very responsive to quick changes in the parameter they are measuring.

It was determined that it would be advantageous to use a sliding 5 sample average. If the pressure were sampled continuously, a complete average would be ready in: 5×20.016=100.090 milliseconds. However, it would not be necessary to wait for all 5 samples to be acquired before a pump control decision was made on the value because just a few samples still mitigates against system anomalies sufficiently. However, sampling the pressure continuously, especially since the pressure sensor 22 places the largest mA load of any component (other than the pump 20) on the system would unnecessarily shorten battery life for too little gain in pump control.

Consequently, a measurement process asynchronous to the pump on/off cycle was used wherein pressure samples are contributed to the average every 100 milliseconds, and if the pump on/off control logic compared the average every 80 milliseconds it would compare it often enough to determine an average affected by the very last contribution that was made to that average. This can be achieved by: starting the process every 100 milliseconds; waiting the 20 milliseconds pressure sensor measurement time; then, measuring the pressure with the A/D.

It is contemplated to synchronize the completion of the AID measurement to the pump on/off control logic exactly but software design best practice says that dependencies between software modules should be avoided where possible and synchronization is not necessary here—the pump on/off control logic (cycle) will see every change any contribution makes to the average.

When the pump 20 has met its target pressure, the AID sampling and comparing process detailed above changes from, for example, a repeat rate of 100 milliseconds to a repeat rate of 1000 milliseconds when the pump has been off for a sufficient amount of time. This further saves battery life at a time when the pressure is not likely to change very much. The period of time the pump 20 has to be off to switch to the slower repeat rate may be, for example, 300 milliseconds; however, it may be any value that was larger than the off period in the pump on/off duty cycle (more later on the duty cycle). Moreover, it should be at least that long to distinguish the “off” that is due to the pump 20 having reached the target pressure and the “off” of the pump control duty cycle. (The 300 milliseconds was utilized as it is a sufficient amount of time for the AID to wait before it samples the battery voltage after the pump is turned off. The battery is only sampled when the pump 20 is turned off because the mA load that the pump places on the system is significantly different than when it is off and battery life is more easily predicted when the pump is not loading the system.)

The pump control on/off duty cycle is preferably 80 milliseconds on and 80 milliseconds off. It was determined that an “on” time of 40 or 80 milliseconds was sufficient for constraining the overshoot for a variety of bandages 12. The 40 milliseconds value constrained it better, yielded end pressures closer to the target pressure, but took longer to get to the target pressure. The “on” times longer than 80 milliseconds created out-of-bounds overshoot for some bandages—no matter what the “off” time was. As mentioned above, the “off” time has an effect on the measurement part of the control I measurement algorithm. Further, plumbing and bandage elasticity, although minor, can contort the average or “steady” pressure for a short while. It is believed that it is advantageous to let such pressure contortions settle before measurement. Moreover, the pressure sensor 22 and AID measurements need time also as noted above.

An “off” time of approximately 30 milliseconds would cover both but it was convenient to use the same 80 milliseconds timer to control both the on time and the off time. Further, letting the system settle longer, for one or more multiples of the 80 milliseconds, would improve the quality of the measurement because it would let the sliding average fill more completely—at the cost of taking longer to get to the end pressure. While these times have been discussed, nothing herein should be construed to limit the present invention to these times.

In use, a user inputs the therapeutic negative pressure into the pumping device. Of course, this can be accomplished with the pumping device having pre-set therapeutic negative pressures and the user merely selects one.

In one or more embodiments of the present invention, a target negative pressure is set slightly less than the therapeutic negative pressure entered by the user into pumping device. In a working example of an embodiment of the invention, the following parameters were used with the object to obtain the therapeutic negative pressure of 40/60/90/125 mmHg within 15 seconds and limit overshoot to +/−10% of the therapeutic negative pressure. However, for the 40 mmHg therapeutic negative pressure, the tolerance is preferably +/−10 mmHg (not +/−10%).

Using a target negative pressure that is set slightly less than the 40/60/90/125 therapeutic negative pressure allows for compensation of the system's tendency to undervalue the actual negative pressure within the bandage. Therefore, the target pressures contemplated to be used with the previously mentioned therapeutic negative pressure values are 34, 54, 85 and 115 mmHg (respectively). Other target pressures can be used depending on the variety of parameters discussed above (pump, bandage, elasticity, etc.) without departing from the spirit of the present invention.

In a device according to one or more embodiments of the present invention, the pump 20 will reasonably quickly achieve the target negative pressure, stop, “top-up” once or twice after a few seconds and transition to the state where it only tops-up every minute or so to compensate for system leaking. The initial one or two top-ups are due to the pressure completely settling.

In sum, in one or more embodiments of the present invention, pressure is sampled every 100 ms; with the sampling by the pressure sensor and AID taking 20.016 milliseconds. Samples go into a sliding 5 element average. The pumping device 10 asynchronously compares the average every 80 milliseconds s (slightly faster than the pressure is sampled). The pumping device 10 turns the pump 20 on for no more than one cycle (80 milliseconds) if pressure is below target pressure. After waiting one off cycle (80 milliseconds), if the pressure is still below the target pressure, the pump 20 is turned on again. These last two steps repeat until the pressure is greater than the target pressure, at which point the pump 20 is left off until the normal leaking in the system reduces the pressure below the target pressure. During the period when the pressure is above the target pressure, starting when the pump 20 has been continuously off for 300 milliseconds, sampling may be reduced to the 1000 milliseconds rate (to extend battery life).

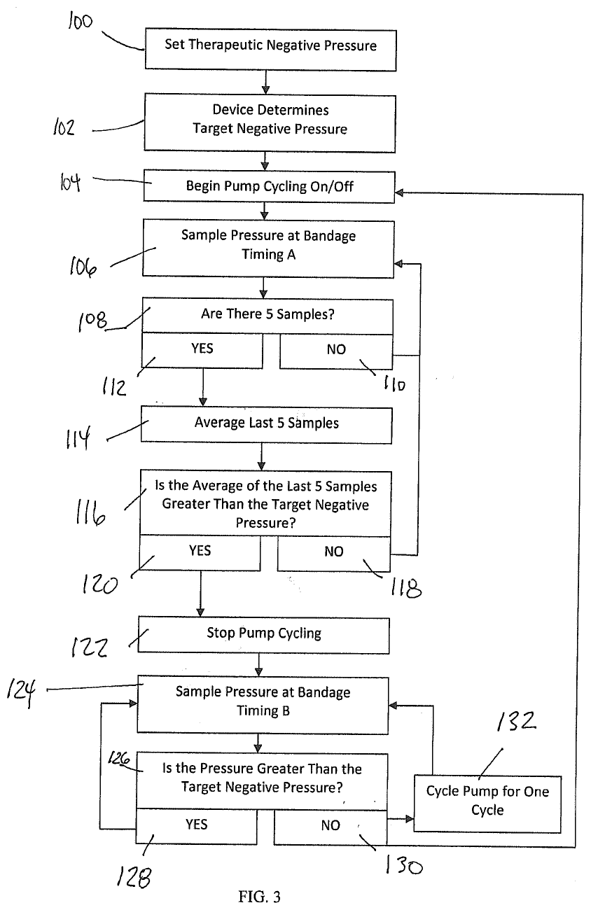

A flow chart for performing the steps of some embodiments is shown in FIG. 3. As shown therein, and as discussed above, a method according to one or more embodiments of the present invention may begin with the setting of a therapeutic negative pressure 100. In response to same, the pumping device will determine a target negative pressure 102. As previously discussed the target negative pressure is less than the therapeutic negative pressure.

At this point, the pumping device will begin to cycle the pump on and off 104. In addition, the pumping device will also begin sampling the pressure at the bandage 106. This sampling is preferably done at a set time. In FIG. 3, this is shown as being at “Timing A.” The pumping device determines if there are at least five samples measured 108. If less than five samples have been collected 100, the pumping device continues to sample the pressure at the bandage 106.

If, however, more than five samples have been collected 112, the pumping device will calculate the average of the last five samples taken 114. The pumping device will then compare the average of the last five samples to the target negative pressure 116. If the average of the last five samples is less than the target negative pressure 118, the pumping device will continue to sample the pressure at the bandage 106.

If the average of the last five samples is greater than the target pressure 120, the pumping device will cease the pump cycling. It is contemplated, although not required, that if the pump is in the middle of a cycle, that specific cycle be completed, before the pumping cycling is stopped. Alternatively, the pump cycling can be immediately stopped when the average is determined to be greater than the target negative pressure.

After the pump cycling has been stopped, the device begins sampling the pressure at the bandage 124. This sampling is conducted, preferably at a predetermined time interval, and as shown, occurs at “Timing B.” As previously mentioned, in order to increase the life of the batteries that may be powering the pumping device, it is preferred that Timing B is greater than Timing A.

Finally, the device continues to compare the sample of the pressure (taken in step 124) with the target negative pressure 126. If the most recent sample is greater than the target negative pressure 128, the device continues to sampling the pressure at the bandage 124.

If the most recent sample is less than the target pressure 130, the device may tum the pump on for one cycle 132, and continue to sample the pressure under the bandage 124. Alternatively, the device can return to cycling the pumping on and off 104 and the subsequent steps previously taken to achieve the target negative pressure. In addition to achieving and maintaining negative pressure, one or more embodiments of the present invention also include a method for detecting a leak. For example, if the target negative pressure has been reached and at least 20 top-ups have occurred at the target negative pressure and the average of the last N (=3) top-up intervals is less than 15 seconds, a leak may be declared by the pump device. The minor leak detection may be cleared if the pump is disabled by the user or the top-up intervals are greater than or equal to 15 seconds.

The 15 seconds time value is related to an unacceptable leak rate (4 top-ups per minute) that was determined to be optimal. The pumping device records the time (since unit reset) of every top-up into a circular array of N elements and checks the top-up interval at every top-up or every 80 milliseconds, whichever occurs first. (The “or 80 milliseconds” facet is more useful for clearing the fault than declaring it.)

The time in the oldest of the array elements is subtracted from the current time and then divided by N to either yield the average of the last N top-ups (or what the average would have been if the 80 milliseconds timer tick that prompted the check was a top-up). An example of setting the fault and clearing the fault is given below:

TABLE ONE
Setting the fault (assume 17 top-ups for
this pressure have already occurred):

| Top-Up # | Time | Note |
|---|---|---|
| 18 | 05:42:23.000 | |
| 19 | 05:42:33.000 | |
| 20 | 05:42:43.000 | Check called due to this top-up. |
| | | System time is 05:42:43. |
| | | System time − oldest top-up time = 00:00:20 |
| | | 00:00:20/3 = 6 |
| | | 6 < 15, fault declared |

The checks called due to the 80 milliseconds timer expiring are not shown in the above because they would have had no effect and their omission makes it easier to understand how the checks due to just the top-ups take place.

TABLE TWO
Clearing the fault (assume a continuation
of the above TABLE ONE)

| Top-Up # | Time | Note |
|---|---|---|
| 18 | 05:42:23.000 | |
| 19 | 05:42:33.000 | |
| 20 | 05:42:43.000 | Check called due to this top-up. |
| | | System time is 05:42:43. |
| | | System time − oldest top-up time = 00:00:20 |
| | | 00:00:20/3 = 6 |
| | | 6 < 15, fault declared |
| | 05:42:43.080 | Check called due to 80 ms timer tick, |
| | | System time is 05:42:43.080 |
| | | System time − oldest top-up time = |
| | | 00:00:20.080 |
| | | 00:00:20.080/3 = 6 |
| | | 6 still less than 15, fault stands |
| | 05:42:43.160 | Similar to the previous, fault stands |
| | etc | No top-ups |
| | etc | No top-ups |
| | 05:43:08.000 | Check called due to 80 ms timer tick |
| | | System time is 05:43:08.000 |
| | | System time − oldest top-up time = 00:00:45 |
| | | 00:00:45/3 = 15 |
| | | 15 is not less than 15, fault cleared. |

In the example above, the fault was cleared within 25 seconds of the last top-up, a fraction of three times the 15 seconds threshold (the threshold used to declare the fault). This is consistent with the goal of clearing the fault quickly if it appears as though the fault has been corrected. It could have been cleared even sooner, as fast as within 15 seconds, if the system time was compared to the newest recorded top-up instead of the oldest—but using more than one top-up time gives greater confidence that the fault has really been cleared.

It is to be understood that additional embodiments of the present invention described herein may be contemplated by one of ordinary skill in the art and that the scope of the present invention is not limited to the embodiments disclosed. While specific embodiments of the present invention have been illustrated and described, numerous modifications come to mind without significantly departing from the spirit of the invention, and the scope of protection is only limited by the scope of the accompanying claims.

What is claimed:

1-17. (canceled)

18. A method of operating a negative pressure wound therapy device, the method comprising, by a controller of the negative pressure wound therapy device:
operating a source of negative pressure of the negative pressure wound therapy device in a first mode to provide, via a fluid flow path, negative pressure to a wound covered by a wound dressing;
determining a pressure in the fluid flow path;
comparing the pressure to a target negative pressure;
at a first time:
determining that the target negative pressure has not been established at the wound; and
in response to determining that the target negative pressure has not been established at the wound, continue operating the source of negative pressure in the first mode;
at a second time:
determining that the target negative pressure has been established at the wound; and
in response to determining that the target negative pressure has been established at the wound, operating the source of negative pressure in a second mode in which less power is consumed than in the first mode,
wherein operating the source of negative pressure in the second mode comprises cycling the source of negative pressure on and off to maintain the target negative pressure; and
indicating a presence of a leak in response to determining that a proportion of time during which the source of negative pressure has been activated over a time period satisfies a threshold indicative of a leak.

19. The method of claim 18, further comprising, in the second mode:
determining that the pressure is more positive than the target negative pressure; and
in response to determining that the pressure is more positive than the target negative pressure, activating the source of negative pressure to provide negative pressure to the wound.

20. The method of claim 19, further comprising activating the source of negative pressure for one cycle.

21. The method of claim 18, further comprising:
in the first mode, determining the pressure at a first sampling rate; and
in the second mode, determining the pressure at a second sampling rate lower than the first sampling rate.

22. The method of claim 18, wherein determining the pressure comprises averaging a plurality of pressures in the fluid flow path to determine an averaged pressure.

23. The method of claim 22, wherein comparing the pressure to the target negative pressure comprises comparing the averaged pressure to the target negative pressure.

24. The method of claim 18, wherein the target negative pressure is between −40 mmHg to −125 mmHg.

25. The method of claim 18, wherein determining the pressure is performed asynchronously with operation of the source of negative pressure.

26. A negative pressure wound therapy apparatus comprising:
a source of negative pressure configured to be fluidically connected, via fluid flow path, to a wound covered by a wound dressing and to provide negative pressure to the wound;
a pressure sensor configured to monitor pressure in the fluid flow path; and
a controller configured to:
operate the source of negative pressure in a first mode;
determine a pressure in the fluid flow path based on one or more readings obtained by the pressure sensor;
compare the pressure to a target negative pressure;
in response to determining that the target negative pressure has not been established at the wound, continue operating the source of negative pressure in the first mode;
in response to determining that the target negative pressure has been established at the wound, operate the source of negative pressure in a second mode in which less power is consumed than in the first mode,
wherein operating the source of negative pressure in the second mode comprises cycling the source of negative pressure on and off to maintain the target negative pressure; and
indicate a presence of a leak in response to determining that a proportion of time during which the source of negative pressure has been activated over a time period satisfies a threshold indicative of a leak.

27. The apparatus of claim 26, wherein the controller is further configured to:
in response to determining that the pressure is more positive than the target negative pressure in the second mode, activate the source of negative pressure to provide negative pressure to the wound.

28. The apparatus of claim 27, wherein the controller is further configured to activate the source of negative pressure for one cycle.

29. The apparatus of claim 26, wherein the controller is further configured to:
in the first mode, determine the pressure by causing the pressure sensor to monitor pressure at a first sampling rate; and
in the second mode, determine the pressure by causing the pressure sensor to monitor pressure at a second sampling rate lower than the first sampling rate.

30. The apparatus of claim 26, wherein the controller is further configured to:
determine an averaged pressure by averaging a plurality of readings of pressure obtained by the pressure sensor.

31. The apparatus of claim 30, wherein the controller is configured to compare the averaged pressure to the target negative pressure.

32. The apparatus of claim 26, wherein the target negative pressure is between −40 mmHg to −125 mmHg.

33. The apparatus of claim 26, wherein the controller is configured to determine the pressure asynchronously with operation of the source of negative pressure.

34. A negative pressure wound therapy apparatus comprising:
a source of negative pressure configured to be fluidically connected, via fluid flow path, to a wound covered by a wound dressing and to provide negative pressure to the wound;
a pressure sensor configured to monitor pressure in the fluid flow path; and
a controller configured to:
operate the source of negative pressure in a first mode;
determine a pressure in the fluid flow path based on averaging a plurality of readings obtained by the pressure sensor;
compare the pressure to a target negative pressure;
in response to determining that the target negative pressure has not been established at the wound, continue operating the source of negative pressure in the first mode; and
in response to determining that the target negative pressure has been established at the wound, operate the source of negative pressure in a second mode in which less power is consumed than in the first mode,
wherein operating the source of negative pressure in the second mode comprises cycling the source of negative pressure on and off to maintain the target negative pressure.

35. The apparatus of claim 34, wherein the controller is further configured to indicate a presence of a leak in response to determining that a proportion of time during which the source of negative pressure has been activated over a time period satisfies a threshold indicative of a leak.

36. The apparatus of claim 34, wherein the controller is further configured to:
in the first mode, determine the pressure by causing the pressure sensor to monitor pressure at a first sampling rate; and
in the second mode, determine the pressure by causing the pressure sensor to monitor pressure at a second sampling rate lower than the first sampling rate.

37. The apparatus of claim 34, wherein the controller is configured to determine the pressure asynchronously with operation of the source of negative pressure.
